(19) United States
(12) Patent Application Publication
(10) Pub. No.: US 2005/0095283 A1
(43) Pub. Date: May 5, 2005

(54) Compositions and methods for topically treating diseases

(75) Inventors: Trevor P. Castor, Arlington, MA (US); Norman D. Weiner, Ann Arbor, MI (US)

Correspondence Address: PERKINS, SMITH & COHEN LLP, ONE BEACON STREET, 30TH FLOOR, BOSTON, MA, 02108, US

(73) Assignee: Aphios Corporation

(21) Appl. No.: 10/940,370

(22) Filed: Sep. 14, 2004

Related U.S. Application Data
(60) Provisional application No. 60/503,321, filed on Sep. 16, 2003.

(51) Int. Cl. A61K 9/127; A61K 33/24; A61K 31/337; A61K 31/704; A61K 31/573

(52) U.S. Cl. 424/450; 514/34; 514/449; 514/171; 424/649; 514/410; 514/575; 514/649; 514/492

(57) ABSTRACT

Described herein are compositions and methods for treating disease. In one aspect, the compositions comprise an anti-neoplastic agent together with a liposome preparation. In another aspect, the compositions of the present invention are directed toward the treatment of cancer. In a particular aspect, the cancer target is Kaposi's sarcoma. The compositions described herein can be topically applied to a subject's integumentary system using liposomal technology.

### RELATED APPLICATIONS

This application claims priority to and the benefit of U.S. Provisional 60/503,321, filed Sep. 16, 2003.

### FIELD OF THE INVENTION

The present invention pertains to compositions and methods for treating disease. In particular, the instant invention employs non-toxic nanosomes for topically delivering one or more pharmacological agents to the integumentary system.

### BACKGROUND OF THE INVENTION

AIDS related Kaposi's sarcoma (AIDS-KS) is a leading cause of death in HIV-immunocompromised patients. Peters et al. (1991) report that deaths attributable to AIDS-KS totaled 14% in 1984 and 32% in 1989. Long term therapy with standard chemotherapeutic regimens has been limited by relatively short durations of response and systemic toxicities. Once therapy is discontinued, the disease typically progresses (Gordon et al., 1995).

Over the past few years, there has been much scientific debate about the cause of KS in AIDS patients. Investigators have demonstrated that a variety of cytokines are produced by AIDS-KS derived spindle cells. It has been hypothesized that these factors act through autocrine and paracrine pathways to induce additional proliferation of spindle cells, as well as to stimulate the angiogenesis that characterizes AIDS-KS histologically (Northfelt, 1994). This model of AIDS-KS histogenesis is the basis for the development and clinical testing of several anti-angiogenic substances.

Cohen (1995) reported on the controversy that AIDS related KS could be caused by a new strain of herpes virus. This finding of KS associated herpes virus (KSHV) has stimulated research on several anti-viral therapies for AIDS-KS. Other therapeutic strategies are to attack the transformed cells with conventional cancer treatments. There are several experimental and palliative treatments for Kaposi's sarcoma (Cohen, 1995), some systemic such as Taxol® (paclitaxel) and liposomal encapsulated anthracyclines, and other localized interventions such as liquid nitrogen cryotherapy and electrocauterization/laser surgery.

Gill et al. (1995) conducted a Phase III clinical and pharmacokinetic evaluation of liposomal daunorubicin (DaunoXome™) for safety, pharmacokinetics and potential efficacy in patients with AIDS-related Kaposi's sarcoma. Forty patients with advanced AIDS-KS received doses of 10 to 60 mg/m² once every 2 weeks. Twenty-two patients who received 50 and 60 mg/m² were assessable for tumor response: 55% (12 of 22) had a partial or clinical complete response. The median survival duration in all patients was 9 months. DaunoXome™ was well tolerated with no significant alopecia, mucositis or vomiting. Anemia and thrombocytopenia were uncommon. Other adverse effects included mild to moderate fatigue, nausea and diarrhea. Even after cumulative doses greater than 1,000 mg/m², no significant declines in cardiac function were observed. In September 1995, the FDA advisory committee recommended approval of a liposomal formulation of the cancer drug daunorubicin as a first-line therapy for Kaposi's sarcoma.

Presant et al. (1993) showed that liposomal daunorubicin is effective even in AIDS-KS patients resistant to other chemotherapy. They assessed efficacy and toxicity of liposomal daunorubicin (50 mg/m² every 2 weeks) in 25 patients with HIV-associated Kaposi's sarcoma of poor prognosis. In 24 evaluated patients, there were 2 complete remissions (8.3%) and 13 partial remissions (54.2%). Five of 11 patients with doxorubicin-resistant Kaposi's sarcoma had partial remissions. Median duration of response was 12 weeks. Quality of life improved after treatment with a response rate of 71% for physical performance and 74% for emotion. Myelosuppression was the most common adverse event. Vomiting, stomatitis and alopecia were rare and mild.

Harrison et al. (1995) conducted a Phase II clinical study of a single-agent liposomally entrapped doxorubicin (Doxil™) against locally advanced cutaneous/systemic AIDS-KS. Thirty-four patients with advanced AIDS-KS were treated with 20 mg/m² of Doxil™ every 3 weeks. An overall response rate of 73.5% (25 of 34) was observed—partial responses of 67.7% (23 of 34) and complete responses of 5.8% (2 of 34). The median time to response was 6 weeks, and the median duration of response was 9 weeks. Toxicity was as follows: 34% with neutropenia (grade >or =3), 9% alopecia (grade 1 only), and 18% nausea and vomiting (grade 1). One patient died of heart failure, which was not considered to be anthracycline-induced. The major toxicity was neutropenia, which appeared to be progressive in patients who receive several cycles of therapy.

In vitro experiments with KS-derived cell cultures, which most likely represent KS spindle cells, suggest that liposomal doxorubicin may cause regression of KS via two different mechanisms: (i) by highly specific inhibition of KS spindle cell proliferation; and (ii) by induction of monocyte chemoattractant protein-1 expression in KS spindle cells, which may result in increased recruitment of phagocytic cells (monocytes/macrophages) into the lesions (Sturzl et al., 1994). These researchers also suggest that the cooperative action of both mechanisms may explain the high efficacy of liposomal doxorubicin in the treatment of AIDS-KS.

Gordon et al. (1995) reports two cases of hand-foot syndrome (HFS) in patients receiving Doxil™ for AIDS-KS. HFS was reversible once treatment stopped; however, treatment cessation resulted in primary disease recurrence. These authors concluded that HFS, which can be debilitating, might be a limiting factor in the prolonged use of Doxil™ for AIDS-KS in some patients. In November 1995, the FDA approved liposomal encapsulated doxorubicin hydrochloride as a second-line therapy for Kaposi's sarcoma.

Gill et al. (1996) compared the safety and efficacy of liposomal daunorubicin with a reference regimen of doxorubicin, bleomycin and vincristine (ABV) in advanced AIDS-related Kaposi's sarcoma in a prospective randomized Phase III trial. Of 232 patients randomized, 227 were treated: 116 with DaunoXome™ and 111 with ABV. The overall response rate was 25% (three CRs and 26 PRs) for DaunoXome™ and 28% (one CR and 30 PRs) for ABV. The difference in response rate was not statistically significant. ABV patients experienced significantly more alopecia and neuropathy. DanuoXome™0 patients experienced more grade 4 neutropenia. Cardiac function remained stable, with no instances of congestive heart failure on either treatment arm.

Currently there exists a need for a new therapeutic regime. This new therapy should be a topically applied agent that has proven efficacy in the treatment of diseases such as Kaposi's sarcoma.

### SUMMARY

The present invention pertains to compositions and methods for treating disease. In one aspect of the present invention, the compositions comprise an anti-neoplastic agent together with a liposome/nanosome. (The terms liposomes and nanosomes are used interchangeably herein unless otherwise indicated.) In one aspect, the compositions of the present invention are directed toward the treatment of cancer. In a particular aspect, the cancer target is Kaposi's sarcoma. The compositions of the instant invention can be topically applied to the subject using liposomal technology.

In one aspect of the present invention, the pharmaceutical agent is an anti-tumor agent. Within a particular aspect of the present invention, the anti-tumor agent is paclitaxel, a compound which disrupts microtubule formation by binding to tubulin to form abnormal mitotic spindles. Paclitaxel is a highly derivatized diterpenoid and can be obtained from the harvested and dried bark of Taxus brevifolia (Pacific Yew) and Taxomyces Andreanae and Endophytic Fungus of the Pacific Yew. Paclitaxel should be understood herein to include prodrugs, analogues and derivatives thereof.

Liposomes are non-toxic, non-antigenic and biodegradable in character since they have the molecular characteristics of mammalian cell membranes. Compounds, such as one or more pharmaceutical agents, are trapped inside the lipid bilayers and/or aqueous core compartment. Encapsulation masks the hydrophobic (water insoluble) nature of the drugs, and permits aqueous, biocompatible formulations to be prepared and administered. Encapsulation also prolongs the drugs' circulation, and for cancer chemotherapy, increases the likelihood that the drug will reach and destroy cancer cells.

### DETAILED DESCRIPTION

The present invention pertains to compositions and methods for treating disease. In one aspect of the present invention, the compositions comprise an anti-neoplastic agent together with a liposome. In one aspect, the compositions of the present invention are directed toward the treatment of cancer. In a particular aspect, the cancer target is Kaposi's sarcoma. The compositions of the instant invention can be topically applied to a subject's integumentary system using liposomal/nanosomal technology.

Kaposi's sarcoma (KS) is the most frequent neoplastic manifestation of HIV infection and is one of the CDC criteria that define an HIV-infected individual as having AIDS. Based upon epidemiological findings, a new member of the γ-herpesvirsus family was elucidated, that being the Kaposi's sarcoma herpesvirus (KSHV) or human harpesvirus 8 (HHV-8). This virus has now been associated with not just KS, but also with a subset of B-cell lymphomas, Castleman's disease and, perhaps multiple myeloma.

The precise mechanism of how the herpes virus participates in tumor development remains enigmatic. There are a number of KSHV genes with human homologues that suggest possible direct effects of the virus or effects at a distance. The nature of the immunologic response to KSHV remains ill defined, but clearly plays an important role in the control of KSHV-related tumors.

Histopathologically, KS lesions are a mixture of different cell types. Endothelial cells are present within the KS lesions, as is a prominent spindle-cell proliferation surrounded by extravasated erythrocytes and macrophages. The cell of origin of the neoplasm is still debated, as is the clonality of the disease.

KS often is diagnosed as having a cutaneous nonblanching red macule. KS lesions can be solitary or disseminated; vary in color from light tan to deep purple; vary in appearance from macules to tumor nodules; or be arranged in a follicular, zosteriform, or linear pattern; they are generally atypical when compared with the lesions of KS occurring in non-HIV-infected individuals.

In one embodiment of the present invention, a composition for treating a disease comprises one or more pharmaceutical agents and a liposome. In one aspect of the instant invention, nanosomes are used to deliver one or more pharmaceutical agents. In one aspect of this embodiment, the pharmaceutical agent is an anti-tumor (or anti-neoplastic) agent. In another aspect of this embodiment, the composition can be prepared consistent with the topical administration of the composition.

Liposomes are non-toxic, non-antigenic and biodegradable in character since they have the molecular characteristics of mammalian cell membranes. Compounds, such as one or more pharmaceutical agents, are trapped inside the lipid bilayers and/or aqueous core compartment. Encapsulation masks the hydrophobic (water insoluble) nature of the drugs, and permits aqueous, biocompatible formulations to be prepared and administered. Encapsulation also prolongs the drugs' circulation, and for cancer chemotherapy, increases the likelihood that the drug will reach and destroy cancer cells. Nanosomal drugs can potentially lead to: (i) enhancement of drug efficacy; (ii) reduction of drug toxicity level; (iii) improved drug stability, and (iv) controlled drug release. Some of the more advanced applications of liposomes have been: systemic treatment of fungal infections and cancer therapy.

Nanosome are lipid vesicles made of membrane-like lipid bilayers separated by aqueous layers. Liposomes have been widely used to encapsulate biologically active agents for use as drug carriers since water- or lipid-soluble substances can be entrapped within the aqueous layers or within the bilayers themselves. There are numerous variables that can be adjusted to optimize this drug delivery system. These include, the number of lipid layers, size, surface charge, lipid composition and the methods of preparation.

Liposomes have been utilized in numerous pharmaceutical applications, including injectable, inhalation, oral and topical formulations, and provide advantages such as controlled or sustained release, enhanced drug delivery, and reduced systemic side effects as a result of delivery localization.

Materials and procedures for forming liposomes are well-known to those skilled in the art and will only be briefly outlined herein. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J. Mol. Biol. 13:238-252 (1965), the entire teaching of which is incorporated herein by reference.

In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979), the entire teaching of which is incorporated herein by reference).

Liposomes can also take the form of unilamellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), the entire teaching of which is incorporated herein by reference, sonication of an aqueous dispersion of phospholipids produces small ULVs having a lipid bilayer surrounding an aqueous solution. Schneider, U.S. Pat. No. 4,089,801, the entire teaching of which is incorporated herein by reference, describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973), the entire teaching of which is incorporated herein by reference, and the ether injection technique of Deamer, et al., Biochim et Biophys Acta 443:629-634 (1976), the entire teaching of which is incorporated herein by reference. These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984), the entire teaching of which is incorporated herein by reference. This detergent removal method involves solubilizing the lipids and additives with detergents by agitation or sonication to produce the desired vesicles.

Papahadjopoulos, et al., U.S. Pat. No. 4,235,871, the entire teaching of which is incorporated herein by reference, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, the entire teaching of which is incorporated herein by reference, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), the entire teaching of which is incorporated herein by reference, these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985), the entire teaching of which is incorporated herein by reference.

Mezei, et al., U.S. Pat. Nos. 4,485,054 and 4,761,288, the entire teaching of which is incorporated herein by reference, also describe methods of preparing lipid vesicles. More recently, Hsu, U.S. Pat. No. 5,653,996, the entire teaching of which is incorporated herein by reference, describes a method of preparing liposomes utilizing aerosolization and Yiournas, et al., U.S. Pat. No. 5,013,497, the entire teaching of which is incorporated herein by reference, describes a method for preparing liposomes utilizing a high velocity-shear mixing chamber. Methods are also described that use specific starting materials to produce ULVs (Wallach, et al., U.S. Pat. No. 4,853,228, the entire teaching of which is incorporated herein by reference) or OLVs (Wallach, U.S. Pat. Nos. 5,474,848 and 5,628,936, the entire teaching of which is incorporated herein by reference).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984), the entire teaching of which is incorporated herein by reference. This and the aforementioned references describing various lipid vesicles suitable for use in the invention are incorporated herein by reference.

The therapeutic compositions can be formulated for topical application. Representative examples include: ethanol; mixtures of ethanol and glycols (e.g., ethylene glycol or propylene glycol); mixtures of ethanol and isopropyl myristate or ethanol, isopropyl myristate and water (e.g., 55:5:40); mixtures of ethanol and eineol or D-limonene (with or without water); glycols (e.g., ethylene glycol or propylene glycol) and mixtures of glycols such as propylene glycol and water, phosphatidyl glycerol, dioleoylphosphatidyl glycerol, Transcutolo, or terpinolene; mixtures of isopropyl myristate and 1-hexyl-2-pyrrolidone, N-dodecyl-2-piperidinone or 1-hexyl-2-pyrrolidone.

Other excipients can also be added to the above, including for example, acids such as oleic acid and linoleic acid, and soaps such as sodium lauryl sulfate (SDS). For a more detailed description of the above, see generally, Hoelgaard et al., J. Contr. Rel. 2:111, 1985; Liu et al., Pharm. Res. 8:938, 1991; Roy et al., J. Pharm. Sci. 83:126, 1991; Ogiso et al., J. Pharm. Sci. 84:482, 1995; Sasaki et al., J. Pharm. Sci. 80:533, 1991; Okabe et al., J. Contr. Rel. 32:243, 1994; Yokomizo et al., J. Contr. Rel. 38:267, 1996; Yokomizo et al., J. Contr. Rel. 42:37, 1996;Mond et al., J. Contr. Rel. 33:72, 1994; Michniak et al., J. Contr. Rel. 32:147, 1994; Sasaki et al., J. Pharm. Sci. 80:533, 1991; Baker & Hadgraft, Pharm. Res. 12:993, 1995; Jasti et al., AAPS Proceedings, 1996; Lee et al., AAPS Proceedings, 1996; Ritschel et al., Skin Pharmacol. 4:235, 1991; and McDaid & Deasy, Int. J. Pharm. 133:71, 1996, the entire teaching of which is incorporated herein by reference.

The topical formulation of an antitumor drug of the present invention reduces the systemic use of these drugs, minimizing blood toxicity levels and improving a patient's quality of life. The nanosomes of the instant invention are ideal vehicles for the topical delivery of the antitumor agents. A variety of non-phospholipid amphiphiles have been shown to form liposomes. Of particular interest are vesicles formed from combinations of glyceryl fatty acid diesters, polyoxyethylene stearyl ether and cholesterol. The physicochemical properties of these and other nonionic liposomal formulations are known to those skilled in the art. The nonionic lipids show very acceptable chemical stability. Furthermore, the raw materials used to form the bilayers have been used extensively as adjuvants in cosmetic products and are considered safe and non-irritating.

The compositions of the present invention include antitumor drugs. Cycle-active agents are drugs that require a cell to be in cycle, i.e., actively going through the cell cycle preparatory to cell division to be cytotoxic. Some of these drugs are effective primarily against cells in one of the phases of the cell. The importance of this designation is that cell cycle-active agents are usually schedule-dependent, and that duration of exposure is as important and usually more important than dose. In contrast, noncell cycle-active agents are usually not schedule-dependent, and effects depend on the total dose administered, regardless of the schedule. Alkylating agents are generally considered to be noncycle active, whereas antimetabolites are prototypes of cycle-active compounds.

An example of cell cycle-active agents are fluoropyrimidines, such as 5-fluorouracil (5-FU) and 5-fluorodeoxyuridine (5-FUdR). 5-FU exerts its cytotoxic effects by inhibition of DNA synthesis, or by incorporation into RNA, thus inhibiting RNA processing and function. The active metabolite of 5-FU that inhibits DNA synthesis through potent inhibition of thymidylate synthase is 5-fluorodeoxyuridylate (5-FdUMP). In rapidly growing tumors, inhibition of thymidylate synthetase appears to be the key mechanism of cell death caused by 5-FU; however, in other tumors, cell death is better correlated with incorporation of 5-FU into RNA. Incorporation of 5-FU into DNA can occur also and may contribute to 5-FU cytotoxicity.

5-FU and 5-FUdR have antitumor activity against several solid tumors, most notably colon cancer, breast cancer, and head and neck cancer. A preparation containing 5-FU is used topically to treat skin hyperkeratosis and superficial basal cell carcinomas.

The major limiting toxicities of 5-FU and 5-FUdR include marrow and GI toxicity. Stomatitis and diarrhea usually occur 4-7 days after treatment. Further treatment is usually withheld until recovery from the toxic side-effects occurs. The nadir of leukopenia and of thrombocytopenia usually occurs 7-10 days after a single dose of a 5-day course. The dose-limiting toxicity to infusions of 5-FUdR through the hepatic artery is transient liver toxicity, occasionally resulting in biliary sclerosis. Less common toxicities noted with 5-FU after systemic administration are skin rash, cerebellar symptoms and conjunctivitis.

Another example of a cell cycle-active agent is methotrexate. This folate antagonist was one of the first antimetabolites shown to induce complete remission in children with ALL. Methotrexate (amethopterin) and aminopterin are analogs of the vitamin folic acid. Methotrexate, and similar compounds, acts by inhibiting the enzyme dihydrofolate reductase. As a consequence of this inhibition, intracellular folate coenzymes are rapidly depleted. These coenzymes are required for thymidylate biosynthesis as well as purine biosynthesis, as such, DNA synthesis is blocked by the use of methotrexate and alike. There is considerable toxicity associated with the use of methotrexate such as myelosuppression and GI distress. An early sign of methotrexate toxicity to the GI tract is mucositis. Severe toxicity can result in diarrhea that is due to small bowel damage that can progress to ulceration and bleeding.

Cytosine arabinoside (ara-C) is an antimetabolite analog of deoxycytidine. In the analog, the OH group is in the β configuration at the 2′ position. This compound was first isolated from the sponge Cryptothethya crypta. Ara-C is the drug of choice for the treatment of acute myelocytic leukemia. Ara-C is converted intracellularly to the nucleotide of triphosphate (ara-CTP) that is both an inhibitor of DNA polymerase and incorporated into DNA. The latter event is considered to cause the lethal action of ara-C. Nausea and vomiting are observed with patients being treated with ara-C.

There is a myriad of other chemotherapeutics considered to be within the scope of this invention. Purine analogs, such as 6-mercaptopurine and 6-thioguanine, define drugs that are also employed in the war against cancer. Hydroxyurea is another drug that is used to treat cancer. Hydroxyurea inhibits ribonucleotide reductase, the enzyme that converts ribonucleotides at the diphosphate level to deoxyribonucleotides. Vinca alkaloids are also involved in the treatment of cancer. The vinca alkaloids include vinblastine, vincristine, and vindesine. Epipodophyllotoxin is a derivative of podophyllotoxin that is used in the treatment of such cancers as leukemia, Hodgkin's, and other cancers.

Alkylating agents such as mechlorethamine, phenylalanine mustard, chlorambucil, ethylenimines and methyl melamines, and alkylsulfonates are employed to treat various cancers.

Nitrosoureas like carmustine, lomustine, and streptozocin are used to treat various cancers and have the ability to readily cross the blood-brain barrier.

Cisplatin (diamino-dichloro-platinum) is a platinum coordination complex that has a broad spectrum antitumor activity. Cisplatin is a reactive molecule and is able to form inter- and intrastrand links with DNA in order to cross-link proteins with the DNA. Carboplatin is another platinum based antitumor drug.

Triazenes like dacarbazine and procarbazine are apart of the antitumor arsenal.

There are antibiotics that have antitumor activity such as anthracyclines, such as doxorubicin, daunorubicin, and mitoxantrone. Other antitumor antibiotics include bleomycin, dactinomycin, mitomycin C, and plycamycin.

There are other antitumor drugs, like asparaginase, that are considered to be within the scope of this invention. These and the other drugs mentioned above all have a toxicity profile that is well known to those skilled in the art.

Other therapeutic agents that can be used in the present invention include cyclophosphamide (cytoxan), melphalan (alkeran), chlorambucil (leukeran), carmustine (BCNU), thiotepa, busulfan (myleran); glucocorticoids such as prednisone/prednisolone, triamcinolone (vetalog); other inhibitors of protein/DNA/RNA synthesis such as dacarbazine (DTIC), procarbazine (matulane); and paclitaxel.

Within a particular embodiment of the present invention, the therapeutic agent is paclitaxel, a compound which disrupts microtubule formation by binding to tubulin to form abnormal mitotic spindles. Briefly, paclitaxel is a highly derivatized diterpenoid (Wani et al., J. Am. Chem. Soc. 93:2325, 1971, the entire teaching of which is incorporated herein by reference) which has been obtained from the harvested and dried bark of Taxus brevifolia (Pacific Yew) and Taxomyces Andreanae and Endophytic Fungus of the Pacific Yew (Stierle et al., Science 60:214-216, 1993, the entire teaching of which is incorporated herein by reference).

“Paclitaxel” (which should be understood herein to include prodrugs, analogues and derivatives such as, for example, TAXOL.RTM., TAXOTERE.RTM., Docetaxel, 10-desacetyl analogues of paclitaxel and 3′N-desbenzoyl-3′N-t-butoxy carbonyl analogues of paclitaxel) can be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; W094/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988, the entire teaching of which is incorporated herein by reference), or obtained from a variety of commercial sources, including for example, Sigma Chemical Co., St. Louis, Mo. (T7402—from Taxus brevifolia).

Representative examples of such paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′, 7-di(sodium 1,2-benzenedicarboxylate, 10-desacetoxy-11,12-dihydrotaxol-10,12(18)-diene derivatives, 10-desacetoxytaxol, Protaxol (2′-and/or 7-O-ester derivatives ), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotal, 10-desacetoxy-7-deoxy-9-deoxotaxol, derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2'succinyltaxol; 2′-(beta-alanyl)-taxol); 2′γ-amino-butyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxy-benzoyl taxol; 2′aliphatic carboxylic acid derivatives of taxol, Prodrugs {2′(N,N-diethylamino-propionyl)taxol, 2′(N,N-dimethyglycyl)taxol, 7(N,N-dimethyl-glycyl)taxol, 2′,7-di-(N,N-dimethylglycyl)taxol, 7(N,N-diethylaminopropionyl)taxol, 2′,7-di(N,N-diethyl-aminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl) taxol, 2′,7-di(L-leucyl)taxol, 2′-(L-isoleucyl)taxol, 7-(L-isoleucyl)taxol, 2′,7-di(L-iso-leucyl)taxol, 2′-(L-valyl)taxol, 7-(L-valyl)taxol, 2′7-di(L-valyl)taxol, 2′-(L-phenylalanyl) taxol, 7-(L-phenylalany)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl) taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-de-acetyltaxol, and taxanes (e.g., baccatin III, cephalomannine, 10-deacetylbaccatin III, brevifoliol, yunantaxusin and taxusin).

Representative examples of microtubule depolymerizing (or destabilizing or disrupting) agents include Nocodazole (Ding et al., J. Exp. Med. 171(3):715-727, 1990; Dotti et al., J. Cell Sci. Suppl. 15:75-84, 1991; Oka et al., Cell Struct. Funct. 16(2): 125-134, 1991; Wiemer et al., J. Cell. Biol. 136(1):71-80, 1997, the entire teaching of which is incorporated herein by reference); Cytochalasin B (Illinger et al., Biol. Cell 73(2-3):131-138, 1991, the entire teaching of which is incorporated herein by reference); Vinblastine (Ding et al., J. Exp. Med. 171(3):715-727, 1990; Dirk et al., Neurochem. Res. 15(11): 1135-1139, 1990; Illinger et al., Biol. Cell 73(2-3): 131-138, 1991; Wiemer et al., J. Cell. Biol. 136(1):71-80, 1997, the entire teaching of which is incorporated herein by reference); Vincristine (Dirk et al., Neurochem. Res. 15(11):1135-1139, 1990; Ding et al., J. Exp. Med. 171(3):715-727, 1990, the entire teaching of which is incorporated herein by reference); Colchicine (Allen et al., Am. J. Physiol. 261(4 Pt. 1):L315-L321, 1991; Ding et al., J. Exp. Med. 171(3):715-727, 1990; Gonzalez et al., Exp. Cell. Res. 192(1):10-15, 1991; Stargell et al., Mol. Cell. Biol. 12(4):1443-1450, 1992, the entire teaching of which is incorporated herein by reference); CI 980 (colchicine analogue) (Garcia et al., Anticancer Drugs 6(4):533-544, 1995, the entire teaching of which is incorporated herein by reference); Colcemid (Barlow et al., Cell. Motil. Cytoskeleton 19(1):9-17, 1991; Meschini et al., J. Microsc. 176(Pt. 3):204-210, 1994; Oka et al., Cell Struct. Funct. 16(2):125-134, 1991, the entire teaching of which is incorporated herein by reference); Podophyllotoxin (Ding et al., J. Exp. Med. 171(3):715-727, 1990, the entire teaching of which is incorporated herein by reference); Benomyl (Hardwick et al., J. Cell. Biol. 131(3):709-720, 1995; Shero et al., Genes Dev. 5(4):549-560, 1991, the entire teaching of which is incorporated herein by reference); Oryzalin (Stargell et al., Mol. Cell. Biol. 12(4): 1443-1450, 1992, the entire teaching of which is incorporated herein by reference); Majusculamide C (Moore, J. Ind. Microbiol. 16(2):134-143, 1996, the entire teaching of which is incorporated herein by reference); Demecolcine (Van Dolah and Ramsdell, J. Cell. Physiol. 166(1):49-56, 1996; Wiemer et al., J. Cell. Biol. 136(1):71-80, 1997, the entire teaching of which is incorporated herein by reference); and Methyl-2-benzimidazolecarbamate (MBC) (Brown et al., J. Cell. Biol. 123(2):387-403, 1993, the entire teaching of which is incorporated herein by reference).

A nanosomal system can be prepared so that the bilayers of each of the resultant formulations are saturated with respect to the drug, in one aspect, paclitaxel. An excess of paclitaxel can be added to the lipid phase during the preparation of the nanosomal formulation. The degree of paclitaxel entrapment can be determined using size exclusion chromatography with, for example, a Sephadex G-75 column.

Nonionic nanosomal formulations can be prepared by using different methods such as hydration of dry film (FILM), reverse-phase evaporation (REV) and melt-stir (MELTING). These methods are described briefly below:

The Film Method involves a predetermined amount of lipid and drug (e.g., paclitaxel) weighed and dissolved in chloroform in a round-bottomed flask. Subsequently, the chloroform is removed using a roto-evaporator at around 40° C. to obtain a thin film. Isotonic 0.05M HEPES buffer, ˜pH 7.4, is then added to the film in the flask and the film is hydrated at around 40° C. for 1 hour with intermittent vortex mixing to produce nanosomal suspensions. The liposome suspensions are then sonicated in a bath sonicator for about 30 minutes at around 20° C.

The Rev Method involves a predetermined amount of lipid and drug weighed and dissolved in ether using a round-bottomed flask. An appropriate amount of isotonic 0.05 M HEPES buffer, ˜pH 7.4, is then added to the same flask. The mixture is then vigorously shaken and sonicated in a bath sonicator for about 30 minutes at around 10° C. to produce an oil-in-water emulsion. The organic solvent in the mixture is then removed under vacuum until foaming has ceased.

The Melting Method involves a predetermined amount of lipid and drug weighed in a scintillation vial, or some similar receptacle. The vial is then capped and heated with stirring, at around 50° C. for GDL (glyceryl dilaurate/cholesterol/polyoxyethylene-10-stearyl ether) systems and around 70° C. for GDS (glyceryl distearate/cholesterol/polyoxyethylene-10-stearyl ether) systems, in a water bath to melt the lipids and to dissolve the drug in the lipid melt. Isotonic 0.05 M HEPES buffer, ˜pH 7.4 preheated in a syringe at around 50° C. is then added to the clear lipid melt and the mixture vigorously stirred with cooling under cold water.

All of the nanosomal suspensions can be examined using inverted light microscopy to assure integrity and quality of the nanosomal preparations. The formulations can be stored at ˜4° C.

PC:CH:PS (mole ratio, 1:0.5:0.1) dehydration-rehydration liposomes (DRV) can be prepared by the method reported by Kirby and Gregoriadis, the entire teaching of which is incorporated herein by reference. Briefly, appropriate amounts of the various lipids, drug, and a-tocopherol (1 percent by weight of the total lipids) are dissolved in chloroform using a round-bottomed flask. The solvent is then removed using a roto-evaporator under vacuum; the flask containing the film is dried overnight in a desiccator to remove any residual solvent. An appropriate amount of isotonic 0.05 M HEPES buffer, ˜pH 7.4, is then added to the film in the flask and the film hydrated at ˜40° C. for about 30 minutes with intermittent vortex mixing. The resultant suspension is then dehydrated at ˜50° C. under vacuum using a roto-evaporator. When the suspension is very viscous, an amount of water equivalent to that removed (determined by weighing the flask and its contents before and after dehydration) is added back to the suspension and rehydrated at ˜40° C. for about 45 minutes. The suspension is then annealed at ˜40° C. for an additional 15 minutes and can be stored at 4° C.

Other carriers that may likewise be utilized to contain and deliver the therapeutic agents described herein include: hydroxypropyl-β-cyclodextrin (Cserhati and Hollo, Int. J. Pharm. 108:69-75, 1994, the entire teaching of which is incorporated herein by reference), liposomes (see e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254, the entire teaching of which is incorporated herein by reference), nanocapsules (Bartoli et al., J. Microencapsulation 7(2): 191-197, 1990, the entire teaching of which is incorporated herein by reference), micelles (Alkan-Onyuksel et al., Pharm. Res. 11(2):206-212, 1994, the entire teaching of which is incorporated herein by reference), implants (Jampel et al., Invest. Ophthalm. Vis. Science 34(11):3076-3083, 1993; Walter et al., Cancer Res. 54:22017-2212, 1994, the entire teaching of which is incorporated herein by reference), nanoparticles (Violante and Lanzafame PAACR), nanoparticles-modified (U.S. Pat. No. 5,145,684, the entire teaching of which is incorporated herein by reference), nanoparticles (surface modified) (U.S. Pat. No. 5,399,363, the entire teaching of which is incorporated herein by reference), taxol emulsion/solution (U.S. Pat. No. 5,407,683, the entire teaching of which is incorporated herein by reference), micelle (surfactant) (U.S. Pat. No. 5,403,858, the entire teaching of which is incorporated herein by reference), synthetic phospholipid compounds (U.S. Pat. No. 4,534,899, the entire teaching of which is incorporated herein by reference), gas borne dispersion (U.S. Pat. No. 5,301,664, the entire teaching of which is incorporated herein by reference), liquid emulsions, foam, spray, gel, lotion, cream, ointment, dispersed vesicles, particles or droplets solid- or liquid-aerosols, microemulsions (U.S. Pat. No. 5,330,756, the entire teaching of which is incorporated herein by reference), polymeric shell (nano- and microcapsule) (U.S. Pat. No. 5,439,686, the entire teaching of which is incorporated herein by reference), taxoid-based compositions in a surface-active agent (U.S. Pat. No. 5,438,072, the entire teaching of which is incorporated herein by reference), emulsion (Tarr et al., Pharm Res. 4:62-165, 1987, the entire teaching of which is incorporated herein by reference), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994, the entire teaching of which is incorporated herein by reference) and implants (U.S. Pat. No. 4,882,168, the entire teaching of which is incorporated herein by reference).

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Pharmaceutically acceptable carriers are commonly added in typical drug formulations. For example, in oral formulations, hydroxypropyl cellulose, colloidal silicon dioxide, magnesium carbonate, methacrylic acid copolymer, starch , talc, sugar sphere, sucrose, polyethylene glycol, polysorbate 80, and titanium dioxide: croscarmeloose sodium, edible inks, gelatin, lactose monohodrate, magnesium stearate, povidone, sodium layryl sulfate, carnuba bax, crospovidone, hydroxypropyl methylcellulose, lactose, microcrystalline cellulose, and other ingredients may be used. For example, galactomannan has been used as a carrier for oral delivery of agents, which are in a non-liquid form. See U.S. Pat. Nos. 4,447,337; 5,128,143; and 6,063,402, the entire teaching of which is incorporated herein by reference.

### EXAMPLE

A) Materials and Methods

Synthetic nonionic lipids, glyceryl distearate (GDS), glyceryl dilaurate (GDL) and polyoxyethylene-10-stearyl ether (POE-10) as well as cholesterol (CH) were obtained from IGI, Inc., Little Falls, NJ. HEPES free acid was obtained from Sigma, St. Louis, Mo. Egg phosphatidylcholine (PC) and phosphatidylserine (PS) were obtained from Avanti Polar Lipids Inc., Alabaster, Ala. α-Tocopherol was obtained from Eastman Kodak, Rochester, N.Y. Azone was obtained from Nelson Research, Irvine, Calif. Daunorubicin and doxorubicin were obtained from Sigma Chemicals, St. Louis, Mo. Paclitaxel was manufactured by Aphios Corporation, Woburn, Mass. Radiolabeled drug (³H-paclitaxel) was obtained from Moravek Biochemicals, Brea, Calif. and DuPont (New England Nuclear), Boston, Mass. Sephadex G-75 was obtained from Pharmacia Inc., Piscataway, N.J. All other chemicals were of analytical grade. Water used was double distilled and deionized using a Millipore Milli-Q system.

(i) Preparation of Paclitaxel Encapsulated Nanosomes/Liposomes

The various nanosomal and liposomal (hereinafter, generally referred to as liposomal) systems were prepared so that the bilayers of each of the resultant formulations would be saturated with respect to paclitaxel. This procedure was used so that comparisons of drug deposition could be made using formulations of equal thermodynamic activity and total lipid concentration (50 mg/mL). Thus, an excess of paclitaxel (prepared as a mixture of radio labeled and cold drug) was added to the lipid phase during the preparation of each of the liposomal formulations. The degree of paclitaxel entrapment was then determined using size exclusion chromatography with Sephadex G-75 columns.

(ii) Nonionic Liposomal Formulations

The nonionic liposomal formulations were prepared by using different methods such as hydration of dry film (FILM), reverse-phase evaporation (REV) and melt-stir (MELTING).

FILM METHOD: Appropriate amounts of the lipids and paclitaxel were accurately weighed and dissolved in chloroform in a round-bottomed flask and chloroform was removed using a roto-evaporator at 40° C. to obtain a thin film. An appropriate amount of isotonic 0.05M HEPES buffer, pH 7.4, was then added to the film in the flask and the film was hydrated at 40° C. for 1 hour with intermittent vortex mixing to produce liposome suspensions. The liposome suspensions were then sonicated in a bath sonicator for 30 minutes at 20° C.

REV METHOD: Appropriate amounts of the lipids and drug were accurately weighed and dissolved in ether in a round-bottomed flask. An appropriate amount of isotonic 0.05 M HEPES buffer, pH 7.4, was then added to the same flask. The mixture was vigorously shaken and sonicated in a bath sonicator for 30 minutes at 10° C. to produce an oil-in-water emulsion. The organic solvent in the mixture was then removed under vacuum until foaming has ceased.

MELTING METHOD: Appropriate amounts of the lipids and drug were accurately weighed in a scintillation vial. The vial was then capped and heated with stirring, at 50° C. for GDL systems and 70° C. for GDS systems, in a water bath to melt the lipids and to dissolve the drug in the lipid melt. Isotonic 0.05 M HEPES buffer, pH 7.4 preheated in a syringe at 50° C. was then added to the clear lipid melt and the mixture was then vigorously stirred with cooling under cold water.

All of the liposome suspensions were examined using inverted light microscopy to assure integrity and quality of the liposomal preparations. The formulations were stored at 4° C. overnight before use in the experiments.

(iii) Phospholipid-Based Liposomal Formulations

PC:CH:PS (mole ratio, 1:0.5:0.1) dehydration-rehydration liposomes (DRV) were prepared by the following method reported by Kirby and Gregoriadis: Briefly, appropriate amounts of the various lipids, drug, trace amount of ³H-drug and a-tocopherol (1 percent by weight of the total lipids) were dissolved in chloroform in a round-bottomed flask. The solvent was then removed using a rotoevaporator under vacuum; the flask containing the film was dried overnight in a desiccator to remove any residual solvent. An appropriate amount of isotonic 0.05 M HEPES buffer, pH 7.4, was then added to the film in the flask and the film was hydrated at 40° C. for 30 minutes with intermittent vortex mixing. The resultant suspension was then dehydrated at 50° C. under vacuum using a roto-evaporator. When the suspension became very viscous, an amount of water equivalent to that removed (determined by weighing the flask and its contents before and after dehydration) was added back to the suspension and rehydrated at 40° C. for 45 minutes. The suspension was then annealed at 40° C. for an additional 15 minutes and stored at 4° C. overnight before use in the diffusion experiments.

(iv) Hydroalcoholic Paclitaxel Solution

A hydroalcoholic solution of drug was prepared in order to serve as control for the liposomal systems. The vehicle consisted of a 60:20:20 (v/v/v) mixture of ethanol: propylene glycol:water. The paclitaxel concentration was 0.5 mg/mL. A trace amount of ³H-drug was also be added to the solution.

(v) In Vitro Diffusion Experiments

Hairless mice were sacrificed and full thickness dorsal skin was excised. Subcutaneous fat was carefully removed using a dull scalpel and appropriate sized pieces of skin was then be mounted on Franz diffusion cells with a surface area of 1.77 sq. cm and a receiver capacity of 7 mL. The skin was exposed to ambient conditions while the dermal side was bathed by a 0.05 M isotonic HEPES buffer, pH 7.4. The receiver solution was stirred continuously using a small Teflon-covered magnet. Care was exercised to remove any air bubbles between the dermis side of the skin and the receiver solution. The temperature of the receiver solution was maintained at 37° C. Following mounting of the skin, 125 μL of the test formulation was applied to the epidermal surface of the hairless mouse skin and carefully spread to achieve complete surface coverage. A minimum of three cells using skin from at least three different animals was used. All experiments were carried out under non-occluded conditions.

At 12 hours, the diffusion set-up was dismantled, and the donor cap was rinsed in 10 niL of buffer followed by a 20 mL methanol rinse. The methanol rinse was allowed to dry in a hood at which time scintillation cocktail was added. The buffer rinse along with the methanol rinse was then assayed for radiolabeled drug. The skin section was then mounted on a board and stripped as follows: A piece of adhesive tape (Scotch Magic Tape, 810, 3M Commercial Office Supply Division, St. Paul, Minn.), 1.9 cm wide and about 6 cm long was used. The tape was of sufficient size to cover the area of skin that is in contact with the test formulation. At least nine strippings were carried out and each strip was analyzed separately for radiolabeled drug. If at the end of nine strippings, the skin did not appear shiny and glossy, additional strippings were carried out until a glossy appearance was seen, ensuring complete removal of the stratum corneum was achieved. The remaining skin and the receiver solution was then assayed for radiolabeled drug.

Assay of the donor rinses, strips, remaining skin and receiver solution was carried out after addition of 15 mL of Ecolite+(ICN Biomedicals, Inc., Irvine, Calif.) to each system using a scintillation counter.

(vi) In Vivo Pharmacokinetc Experiments

Hairless mice (45-60 days old) were anesthetized with sodium pentobarbital (60 mg/Kg, i.p.). An open circular glass donor cap (12 mm inner diameter and 8 mm high) was glued to the dorsal skin surface of the mouse using Extra Strength Krazy Glue (Krazy Glue, Inc., Itasca, Ill.). Eighty μL of the test formulation was applied onto each site within the donor compartment. Two sites per animal were used and, since systemic absorption was monitored, both sites were treated with the same test formulation. All experiments were carried out under non-occluded conditions. Periodic additional anesthetization was carried out approximately every one and half hours. A minimum of three animals was used per formulation per time point.

At the determined time point, the animals were sacrificed by a lethal injection of pentobarbital. The skin was then carefully excised and the bladder harvested. The donor caps were detached and thoroughly washed with 15 mL of buffer followed by three 5 niL methanol rinses. The methanol rinses were allowed to dry before scintillation cocktail was added. The skin section was then be mounted on a wooden board and stripped as described earlier. Stripping was carried out until the skin appeared shiny and glossy, usually about 20 times. The remaining skin and bladder along with the donor rinses and strips were then assayed separately for drug content using a scintillation counter after addition of 15 mL of Ecolite+scintillation cocktail (ICN Biomedicals, Inc., Irvine, Calif.).

(vii) In vitro Efficacy Experiments

Cells: KSC1, KSC2 and KSC3 cell lines were kindly provided to Aphios Corp. by Dr. Jacques Corbeil (1994) of the Department of Medicine at the University of San Diego, San Diego, Calif. [Kaposi's sarcoma cell cultures were derived from explants of cutaneous biopsies of KS lesions from AIDS-KS patients and established as long term cultures.] Cells were maintained in Dulbecco's modified media, high glucose, devoid of L-valine and containing D-valine in order to inhibit fibroblast growth. HUVEC (human umbilical vein endothelial cells) were used as a control to assess the effects of the drug formulations on normal cells. HUVEC were obtained from Clonetics (San Diego, Calif.), and used and maintained according to manufacturer's directions.

Cell proliferation assay: Cells were plated in 96-well plates at a density of 3,000-5,000 cells per well and allowed to adhere for 24 hours. Drug formulations were added to the cells and the plates were incubated for 2 days. On the third day, cell density was assayed using the CellTiter 96 Aqueous Cell Proliferation Assay from Promega (Madison, Wis.).

In Vivo Efficacy Experiments: 2×10⁶ KSC were transplanted subcutaneously on day 0 onto the backs of male BALB/c nu/nu athymic mice. Drug formulations were administered topically daily for 4-5 days. On day 6 the mice were sacrificed in a humane manner and the KS-like lesions excised from the skin, mounted histologically, stained and examined for differences in morphology and cell characteristics against control mice treated with empty liposomes.

B) Experimental Results

The deposition of paclitaxel following topical in vitro or in vivo application of various formulations was determined. The formulations tested included: (i) two novel nonionic systems, glyceryl dilaurate/cholesterol/polyoxyethylene-10-stearyl ether (GDL), glyceryl distearate/cholesterol/polyoxyethylene-10-stearyl ether (GDS); (ii) a phospholipid-based system (PC) and (iii) a hydroalcoholic solution (HA).

A factorial design used in to optimize the topical formulation of a relatively hydrophobic anticancer drug. In this design, four factors were used in combination at three different levels. Thus, the design incorporated different methods of preparation, lipid composition effects, total lipid concentration and total drug concentration variations. The different parameters utilized in the factorial design are summarized in Table 1.

TABLE 1
Factorial Design for Optimizing GDL Liposomal Formulations

| | | B | | |
|---|---|---|---|---|
| | | Lipid | C | D |
| | A | Composition | Lipid | Drug |
| Factors | Preparation | (GDL:CH: | Concentration | Concentration |
| Level | Method | POE-10) | (mg/ml) | (mg/ml) |
| 1 | Film | 55:19:26 | 100 | 1.0 |
| 2 | REV | 61:11:28 | 150 | 0.75 |
| 3 | Melting | 61:11:28 | 50 | 0.5 |

The formulations derived from this factorial design are summarized in Table 2.

TABLE 2
Summary of Formulations Derived from the Factorial Design

| Factors | GDL-1 | GDL-2 | GDL-3 | GDL-4 | GDL-5 | GDL-6 | GDL-7 | GDL-8 | GDL-9 |
|---|---|---|---|---|---|---|---|---|---|
| A | 1 | 1 | 1 | 2 | 2 | 2 | 3 | 3 | 3 |
| B | 1 | 2 | 3 | 1 | 2 | 3 | 1 | 2 | 3 |
| C | 1 | 2 | 3 | 2 | 3 | 1 | 3 | 1 | 2 |
| D | 1 | 2 | 3 | 3 | 1 | 2 | 2 | 3 | 1 |

The formulations tested are listed in Table 3.

TABLE 3
Summary of Paclitaxel Liposomal and Hydroalcoholic Formulations

| | Drug Conc. | Lipid Conc. | Composition | Preparation | |
|---|---|---|---|---|---|
| Formulation | (mg/ml) | (mg/ml) | GDX:CH:POE-10 | Method | Code |
| GDL Liposome | 0.5 | 50 | 58:15:27 | Melting | GDL |
| GDS Liposome | 0.5 | 50 | 58:15:27 | Melting | GDS |
| PC Liposome | 0.5 | 50 | 1:0.5:0.1** | DRV | PC |
| Hydroalcoholic | 0.5 | N/A | 6:2:2*** | N/A | HA |
| Solution | | | | | |
| GDL Liposome 1 | 1.0 | 100 | 55:19:26 | Film | GDL-1 |
| GDL Liposome 2 | 0.75 | 150 | 61:11:28 | Film | GDL-2 |
| GDL Liposome 3* | 0.5 | 50 | 61:11:28* | Film | GDL-3 |
| GDL Liposome 4 | 0.5 | 150 | 55:19:26 | REV | GDL-4 |
| GDL Liposome 5 | 1.0 | 50 | 61:11:28 | REV | GDL-5 |
| GDL Liposome 6* | 0.75 | 100 | 61:11:28* | REV | GDL-6 |
| GDL Liposome 7 | 0.75 | 50 | 55:19:26 | Melting | GDL-7 |
| GDL Liposome 8 | 0.5 | 100 | 61:11:28 | Melting | GDL-8 |
| GDL Liposome 9* | 1.0 | 150 | 61:11:28* | Melting | GDL-9 |
| GDL Liposome 10 | 0.5 | 50 | 61:11:28 | Film | GDL-10 |
| GDL Liposome 11 | 0.5 | 50 | 58:15:27* | Film | GDL-11 |

*contains 0.5% by volume of azone.
**PC:CH:PS (mole ratio).
***ethanol:propylene glycol:water (v/v/v)

The results of deposition studies after topical in vitro application of various GDL liposomal formulations are presented in Table 4. This table summarizes the distribution of paclitaxel (expressed as percent of applied dose±S.D.) in various strata of hairless mouse skin 12 hours. In all cases, total recovery was greater than 90%.

TABLE 4
Distribution of Paclitaxel after Topical In Vitro Application of
GDL Liposomes

| Formulation | Total Strips | Living Skin Strata | Receiver |
|---|---|---|---|
| GDL-1 | 69.8 ± 6.3 | 1.18 ± 0.28 | 1.69 ± 0.29 |
| GDL-2 | 57.6 ± 7.5 | 0.69 ± 0.10 | 1.01 ± 0.21 |
| GDL-3* | 66.8 ± 8.4 | 1.52 ± 0.09 | 1.92 ± 0.15 |
| GDL-4 | 59.2 ± 7.5 | 0.61 ± 0.14 | 0.87 ± 0.10 |
| GDL-5 | 53.1 ± 1.3 | 0.96 ± 0.06 | 1.93 ± 0.07 |
| GDL-6* | 72.8 ± 4.0 | 0.79 ± 0.03 | 1.87 ± 0.01 |
| GDL-7 | 60.2 ± 5.7 | 0.62 ± 0.07 | 1.09 ± 0.19 |
| GDL-8 | 67.2 ± 13.8 | 0.98 ± 0.27 | 1.93 ± 0.21 |
| GDL-9* | 64.2 ± 1.5 | 0.59 ± 0.02 | 1.13 ± 0.15 |
| GDL-10 | 60.5 ± 9.1 | 1.23 ± 0.16 | 1.78 ± 0.19 |
| GDL-11* | 61.6 ± 5.1 | 1.41 ± 0.11 | 1.97 ± 0.33 |

*contains 0.5% by volume of azone

Table 5 shows the distribution of paclitaxel (expressed as percent of applied dose±S.D.) in various strata of hairless mouse skin 4, 8 and 12 hours after in vivo topical application of various formulations. In all cases, total recovery was greater than 90%. It is clear that the GDL liposomal system is significantly superior in facilitating delivery of paclitaxel into the living skin strata than either the GDS liposome formulation or the PC-based liposomal formulation or HA. The amounts of paclitaxel in the living skin strata or in urine was found to be significantly higher for the GDL formulation compared to the others at all time points tested (p<0.05).

TABLE 5
In vivo Kinetics of Uptake of Paclitaxel Observed in Hairless Mouse

| Formu- | Total Strips | | | Living Skin Strata | | | Urine | | |
|---|---|---|---|---|---|---|---|---|---|
| lation | 4 h | 8 h | 12 h | 4 h | 8 h | 12 h | 4 h | 8 h | 12 h |
| GDL | 47.9 ± 4.0 | 50.9 ± 3.9 | 51.2 ± 4.6 | 0.65 ± 0.17 | 0.69 ± 0.01 | 0.72 ± 0.09 | 0.22 ± 0.02 | 1.00 ± 0.03 | 1.37 ± 0.12 |
| GDS | 54.5 ± 1.6 | 52.0 ± 1.8 | 48.3 ± 1.2 | 0.07 ± 0.01 | 0.09 ± 0.01 | 0.11 ± 0.03 | 0.14 ± 0.02 | 0.32 ± 0.04 | 0.50 ± 0.08 |
| PC | 50.1 ± 7.8 | 55.4 ± 7.3 | 71.3 ± 4.6 | 0.07 ± 0.01 | 0.06 ± 0.02 | 0.06 ± 0.01 | 0.12 ± 0.02 | 0.32 ± 0.03 | 0.48 ± 0.07 |
| HA | 51.6 ± 2.3 | 70.4 ± 10.3 | 62.5 ± 1.6 | 0.08 ± 0.02 | 0.11 ± 0.05 | 0.13 ± 0.02 | 0.20 ± 0.03 | 0.38 ± 0.02 | 0.59 ± 0.03 |
| GDL-3 | 46.9 ± 2.7 | 58.0 ± 4.1 | 54.4 ± 10.7 | 0.94 ± 0.12 | 0.99 ± 0.25 | 1.07 ± 0.39 | 0.32 ± 0.04 | 1.32 ± 0.04 | 1.92 ± 0.09 |

Table 6 shows the distribution of paclitaxel (expresed as percent of applied dose±S.D.) in various strata of hairless mouse skin 12 hours after topical in vitro and in vivo application of various formulations. In all cases, total recovery was greater than 90%.

TABLE 6
In vitro and In vivo Distribution of Liposomal Paclitaxel
in Various Strata of Hairless Mouse

| | | | | | |
|---|---|---|---|---|---|
| | GDL | GDL-3 | GDS | PC-based | HA |
| Compartment | Liposomes | Liposomes | Liposomes | Liposomes | Solution |

In vitro

| | | | | | |
|---|---|---|---|---|---|
| Total Strips | 65.4 ± 4.5 | 66.7 ± 8.4 | 77.5 ± 1.2 | 59.7 ± 5.6 | 52.3 ± 4.2 |
| Living Skin | 1.10 ± 0.09 | 1.52 ± 0.12 | 0.16 ± 0.06 | 0.05 ± −O.03 | 0.22 ± 0.08 |
| Strata | | | | | |
| Receiver | 2.23 ± 0.10 | 2.82 ± 0.46 | 0.78 ± 0.06 | 0.45 ± 0.02 | 0.85 ± 0.03 |

In vivo

| | | | | | |
|---|---|---|---|---|---|
| Total Strips | 51.2 ± 4.1 | 54.4 ± 10.7 | 48.3 ± 1.2 | 71.2 ± 4.6 | 62.4 ± 1.6 |
| Living Skin | 0.72 ± 0.10 | 1.07 ± 0.02 | 0.11 ± 0.03 | 0.05 ± 0.01 | 0.13 ± 0.02 |
| Strata | | | | | |
| Urinary Bladder | 1.37 ± 0.12 | 1.92 ± 0.09 | 0.50 ± 0.08 | 0.48 ± 0.07 | 0.59 ± 0.03 |

It is clear from Table 6 that the efficacy of transport of paclitaxel into and across hairless mouse skin, when compared at the same drug loading, is in the order GDL liposomal system>>HA>GDS liposomes>PC-based liposomal system. The uptake in the living skin strata was roughly 5 times higher from GDL liposomes compared to the HA solution in both in vitro and in vivo experiments. GDS liposomes and PC-based liposome formulations do not appear to be as efficient as the hydroalcoholic solution.

Although the analyses of organs were not carried out, the amounts of paclitaxel in the urinary bladder also reflect the greater efficiency of GDL liposomes compared to the HA or GDS liposomes or PC-based liposomes, and parallels the amounts of paclitaxel found in receiver solution in vitro from GDL liposomal formulation which is higher than that from HA or GDS liposomal formulation or PC-based liposomal formulation. An excellent correlation is shown between the amounts of paclitaxel in the living skin strata and between the amounts of paclitaxel in urinary bladder and receiver compartment at 12 hours following in vitro or in vivo application (r²=0.997 and 0.98 respectively). The results corroborate the validity of in vitro experiments.

The results of physical stability studies of the GDL formulations after one month are presented in Table 7.

TABLE 7
Examination of Physical Stability of GDL-Liposomal
Formulations of Paclitaxel

| | | | View Under Microscope | | |
|---|---|---|---|---|---|
| Formulation | Entrapment | | | Aggre- | |
| Code | (%) | Appearance | Crystal | gation | Integrity |
| GDL-1 | 22.78 | 1 | 1 | 1 | 0.5 |
| GDL-2 | 16.76 | 1 | 1 | 1 | 0.5 |
| GDL-3* | 75.01 | 0 | 0 | 0 | 0 |
| GDL-4 | 19.62 | 0.5 | 1 | 0.5 | 1 |
| GDL-5 | 25.39 | 0.5 | 1 | 1 | 1 |
| GDL-6* | 53.53 | 0.5 | 0 | 1 | 0.5 |
| GDL-7 | 10.02 | 1 | 0.5 | 1 | 1 |
| GDL-8 | 4.14 | 1 | 1 | 1 | 0.5 |
| GDL-9* | 37.09 | 0.5 | 0 | 1 | 0.5 |
| GDL-10 | 15.41 | 1 | 1 | 0.5 | 0.5 |
| GDL-11* | 66.71 | 0 | 0 | 0 | 0 |

*contains 0.5% by volume of azone
0 - stable,
0.5 - intermediate,
1 - unstable.

The formulation factors and results from the factorial design experiments are summarized in Table 8. It is evident from the Table 8 that formulations containing a zone not only exhibited enhanced deposition profiles but also allowed higher entrapment of paclitaxel and was the most stable.

TABLE 8
Summary of Formulation Factors and Factorial
Design Experiments

| | | | | | % in | % | |
|---|---|---|---|---|---|---|---|
| Formulation | A | B | C | D | Dermis | Entrapment | Stability |
| GDL-1 | 1 | 1 | 1 | 1 | 1.18 | 22.8 | 3.5 |
| GDL-2 | 1 | 2 | 2 | 2 | 0.69 | 16.8 | 3.5 |
| GDL-3* | 1 | 3 | 3 | 3 | 1.52 | 75.1 | 0.0 |
| GDL-4 | 2 | 1 | 2 | 3 | 0.61 | 19.6 | 3.0 |
| GDL-5 | 2 | 2 | 3 | 1 | 0.96 | 25.4 | 3.5 |
| GDL-6* | 2 | 3 | 1 | 2 | 0.79 | 53.5 | 2.0 |
| GDL-7 | 3 | 1 | 3 | 2 | 0.62 | 10.0 | 3.5 |
| GDL-8 | 3 | 2 | 1 | 3 | 0.98 | 4.1 | 3.5 |
| GDL-9* | 3 | 3 | 2 | 1 | 0.59 | 37.1 | 2.0 |

A statistical comparison of results is shown in Table 9. These results indicate that of all the factors examined, the most significant one involved the addition of a zone to the GDL liposome formulation.

TABLE 9
Statistical Comparison of Results from Factorial Design Experiments

| | % in Living Skin | | | | % Entrapment | | | | Stability | | | |
|---|---|---|---|---|---|---|---|---|---|---|---|---|
| | A | B | C | D | A | B | C | D | A | B | C | D |
| K₁ⱼ | 3.39 | 2.4 | 2.95 | 2.73 | 114.54 | 52.4 | 80.45 | 85.26 | 6 | 10 | 9 | 9 |
| K₂ⱼ | 2.36 | 2.6 | 1.89 | 2.1 | 98.54 | 46.29 | 73.47 | 80.29 | 8.5 | 10 | 8 | 8.5 |
| K₃ⱼ | 2.19 | 2.9 | 3.1 | 3.11 | 51.23 | 165.62 | 110.39 | 98.76 | 9 | 4 | 7 | 6.5 |
| Rⱼ | 1.2 | 0.4 | 1.19 | 1.01 | 63.31 | 119.33 | 36.92 | 18.47 | 2.5 | 6 | 2 | 2.5 |
| P | | | | | <0.01 | | | | <0.05 | | | |

*Kᵢⱼ is the sum of experimental results for systems with factor j at level i.
R is the difference between largest and smallest Kij for a given j.
i = 1, 2, 3; j = A, B, C, D.

(i) In Vivo Treatment of Tumors Induced by KS Y-1 Cells with Topical Preparation

To demonstrate the effect of the developed topical formulation GDL-3, KS tumors [range from 0.3×0.5-0.4×0.5 mm] were induced by the inoculation of KS Y-1 cells in immunodeficient mice. The mice were then treated slowly and consistently by Alzet osmotic pumps —0.05 mg—and another group treated with 0.1 mg/daily for 1 week with the topical formulation. Per Table 10, the PBS buffer-treated lesions in three mice showed continued tumor progression [from 1.4×2 to 1.5×2.3 mm], whereas three mice treated with the topical formulation showed almost-complete regression confirming the anti-KS effect of the topical treatment observed in this study (see FIG. 1ABC).

TABLE 10
In Vivo Treatment of Tumors Induced by KS Y-1 Cells
with Topical Preparation

| | Size of KS Y-1Tumors | |
|---|---|---|
| Protocol | Untreated | Post Therapy |
| A: Rx PBS | 0.4 × 0.4 mm | 1.5 × 2.3 mm |
| | 0.3 × 0.5 mm | 1.4 × 2.0 mm |
| | 0.4 × 0.3 mm | 1.5 × 2.1 mm |
| B: Rx Topical: 0.05 mg/d/7d | 0.4 × 0.4 mm | No measurable tumors |
| | | [n = 3] |
| | 0.4 × 0.5 mm | |
| | 0.3 × 0.3 mm | |
| C: Rx Topical: 0.1 mg/d/7d | 0.3 × 0.5 mm | No measurable tumors |
| | | [n = 3] |
| | 0.3 × 0.3 mm | |
| | 0.3 × 0.4 mm | |

While this invention has been particularly shown and described with references to specific embodiments, it will be understood by those skilled in the art that various changes in form and details may be made therein without departing from the spirit and scope of the invention as defined by the appended claims.

1. A method of treating disease, comprising topical administration to a subject in need thereof a therapeutically effective amount of a pharmaceutical agent formulated in a liposomal preparation.

2. The method of claim 1, wherein said disease is a cancer.

3. The method of claim 2, wherein said cancer is Kaposi's sarcoma.

4. The method of claim 1, wherein said pharmaceutical agent is selected from the group consisting of paclitaxel, 5-FU, 5-FUdR, methotrexate, ara-C, 6-mercaptopurine, 6-thioguanine, hydroxyurea, mechlorethamine, phenylalanine mustard, chlorambucil, ethylenimines, methyl melamines, carmustine, lomustine, streptozocin, Cisplatin, Carboplatin, dacarbazine, procarbazine, doxorubicin, daunorubicin, mitomycin C, plycamycin, cyclophosphamide, melphalan, chlorambucil, carmustine, thiotepa, busulfan, prednisone, prednisolone, triamcinolone, and derivatives thereof.

5. The method of claim 4, wherein said pharmaceutical agent is paclitaxel and derivatives thereof.

6. The method of claim 5, wherein said derivatives are selected from the group consisting of 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol, phosphonooxy and carbonate derivatives of taxol, taxol 2′,7-di(sodium 1,2-benzene-dicarboxylate, 10-desacetoxy-11,12-dihydrotaxol-10,12(18)-diene derivatives, 10-desacetoxytaxol, Protaxol (2′-and/or 7-O-ester derivatives ), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotal, 10-desacetoxy-7-deoxy-9-deoxotaxol, derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol), 2′γ-amino-butyryltaxol formate, ethylene glycol derivatives of 2′-succinyltaxol, 2′-glutaryltaxol, 2′-(N,N-dimethylglycyl) taxol, 2′-(2-(N,N-dimethylamino)propionyl)taxol, 2′orthocarboxy-benzoyl taxol; 2′aliphatic carboxylic acid derivatives of taxol, Prodrugs {2′(N,N-diethylamino-propionyl)taxol, 2′(N,N-dimethyglycyl)taxol, 7(N,N-dimethyl-glycyl)taxol, 2′,7-di-(N,N-dimethylglycyl)taxol, 7(N,N-diethylaminopropionyl)taxol, 2′,7-di(N,N-diethyl-aminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl) taxol, 2′,7-di(L-leucyl)taxol, 2′-(L-isoleucyl)taxol, 7-(L-isoleucyl)taxol, 2′,7-di(L-iso-leucyl)taxol, 2′-(L-valyl)taxol, 7-(L-valyl)taxol, 2′7-di(L-valyl)taxol, 2′-(L-phenylalanyl) taxol, 7-(L-phenylalany)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl) taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-de-acetyltaxol, and taxanes.

7. The method of claim 1, wherein said liposomal preparation comprises a lipid bilayer.

8. The method of claim 1, wherein said liposomal preparation encapsulates said pharmaceutical agent.

9. The method of claim 8, wherein said encapsulation is within an aqueous layer of said liposomal preparation.

10. The method of claim 1, wherein said liposomal preparation is selected from the group consisting of an ULV, MLV and OLV.

11. The method of claim 10, wherein said liposomal preparation is an ULV.

12. The method of claim 10, wherein said liposomal preparation is an MLV.

13. The method of claim 10, wherein said liposomal preparation is OLV.

14. The method of claim 1, wherein said liposomal preparation is multivesicular.

15. The method of claim 1 further comprising one or more excipients.

16. The method of claim 15, wherein said excipients are selected from the group consisting of alcohols, glycols, isopropyl myristate, water, mixtures thereof, eineol, D-limonene (with or without water), ethylene glycol or propylene glycol, phosphatidyl glycerol, dioleoylphosphatidyl glycerol, Transcutolo, or terpinolene; mixtures of isopropyl myristate and 1-hexyl-2-pyrrolidone, N-dodecyl-2-piperidinone or 1-hexyl-2-pyrrolidone, and sodium lauryl sulfate.

17. The method of claim 1, wherein said liposomal preparation is a nonionic nansomal formulation.

18. A method of treating cancer, comprising topical administration to a subject in need thereof a therapeutically effective amount of a pharmaceutical agent formulation in a liposomal preparation.
